(19) United States
(12) Patent Application Publication
(10) Pub. No.: US 2015/0032012 A1
(43) Pub. Date: Jan. 29, 2015

(54) Non-invasive Blood Pressure Measurement System and Methods of Use

(71) Applicants: Stephen Marinello, Lubeck (DE); Donald Fournier, Georgetown, MA (US)

(72) Inventors: Stephen Marinello, Lubeck (DE); Donald Fournier, Georgetown, MA (US)

(73) Assignee: Draeger Medical Systems, Inc., Andover, MA (US)

(21) Appl. No.: 14/379,268

(22) PCT Filed: Feb. 17, 2012
(86) PCT No.: PCT/US2012/025725

§ 371 Date: Aug. 15, 2014

(51) Int. Cl. A61B 5/022; A61B 5/00; A61B 5/0215

(52) U.S. Cl. CPC A61B 5/02225; A61B 5/0215; A61B 5/0022
USPC 600/486; 600/490

(57) ABSTRACT

Disclosed is a system that includes a signal acquisition circuit to acquire a cuff pressure signal using an inflatable cuff, and to generate an oscillometric signal from the cuff pressure signal. The system also includes a user interface to enter one or more patient-specific detection threshold values, a memory to store the cuff pressure signal, the oscillometric signal, and the one or more patient-specific detection threshold values. The system also includes a microprocessor operatively coupled to the signal acquisition circuit, the user interface, and the memory. The microprocessor includes at least one algorithm to use the cuff pressure signal, the oscillometric signal, and the entered one or more patient-specific detection threshold values to calculate patient-specific readings of at least one of mean arterial pressure, systolic blood pressure and diastolic blood pressure. Related apparatus, systems, methods and/or articles are described.

### TECHNICAL FIELD

The subject matter described herein relates generally to the field of medical devices, and more particularly to devices, systems, articles, and methods used to improve the accuracy of non-invasive blood pressure measurements.

### BACKGROUND

Invasive and non-invasive blood pressure (NIBP) measuring methods known in the art each have advantages and disadvantages. Invasive blood pressure measurement involves penetrating the arterial wall in order to take a direct measurement of blood pressure using a pressure sensor probe. However, such a measurement is invasive and prone to certain risks to the patient such as infection, arterial embolism or occlusion. Moreover for many patients, such as those in emergency situations where there is insufficient time to insert a catheter probe, or those in shock and therefore the artery susceptible to collapse, the insertion of a catheter may be either impractical or contraindicated. Therefore in these situations, and generally, noninvasive blood pressure measurement methods are desirable over invasive measurements although they may yield somewhat lower accuracy, as will be described below.

One form of NIBP measurement is the auscultatory method, which involves using a sphygmomanometer and stethoscope. An inflatable cuff is positioned around the upper arm of a patient roughly level with the patient's heart. The cuff, attached to a manometer, is inflated until the brachial artery at the elbow is completely occluded. The stethoscope is used to listen to the brachial artery as the pressure in the cuff is slowly released. When blood again starts to flow in the artery, the turbulent flow acting against the arterial walls vibrates creating a whooshing noise known as Korotkoff sounds. The first of five phases of Korotkoff sounds appears at the point when the pressure within the cuff is equivalent to the systolic or peak pressure within the blood vessel. The pressure in the cuff is then further released until laminar blood flow is restored and no sound can be heard which represents the fifth Korotkoff phase, indicative of the diastolic arterial pressure. Because the primary embodiment of the auscultatory method involves a stethoscope and manual interpretation of the Korotkoff sounds by a clinician, it is best suited for periodic as opposed to continuous NIBP readings.

The oscillometric method is another NIBP measurement method that involves the electronic observation of oscillations in the sphygmomanometer cuff pressure caused by the changes in arterial flow resulting from inflating and deflating the cuff. The cuff pressure oscillations are observed using a pressure sensor or transducer and electronics to automatically interpret the oscillations. The inflatable cuff suitably located on the limb of a patient is inflated to a predetermined pressure above the patient's estimated systolic pressure. The cuff pressure is then gradually reduced over a relatively short period of time in predetermined decrements to below diastolic pressure. At each level, the oscillations in the cuff are monitored by the transducer. When blood flow is obstructed and when blood flow is unimpeded, the cuff pressure will be relatively constant and no oscillations present. When some blood flow is present, but restricted, the cuff pressure monitored by the pressure transducer will vary with the cyclic expansion and contraction of the brachial artery generating oscillation signals. As the decrementing continues, the peak amplitudes of the oscillations will normally increase from a lower level to a relative maximum, and thereafter will decrease. These amplitudes form an oscillometric envelope for the patient. The cuff pressure at which the oscillations have a maximum value has been found to be representative of the mean arterial pressure (MAP).

The oscillometric method provides certain advantages in that readings can be taken on a nearly continuous basis with minimal to no risk to the patient as compared to the invasive method. The oscillometric method can also be performed automatically with minimal effort as compared to the auscultatory method, and unlike the auscultatory method, which can directly obtain only systolic and diastolic measurements, can obtain systolic, diastolic and mean arterial pressure measurements. Further, the readings can be performed by a lay person, and because they are automatic, in hospital settings serve as a surveillance tool to monitor blood pressure continuously, providing a trend of any changes in blood pressure and/or alerting a clinician of any significant change.

The benefit of an oscillometric method notwithstanding, one disadvantage is that the values of systolic and diastolic pressure are not actually measured from the raw data, but are computed using an algorithm programmed into the device. Whereas the MAP is directly determined by the maximum amplitude of the oscillations obtained on each individual patient, the systolic and diastolic pressures are derived as predetermined fractions or thresholds of MAP. Moreover the thresholds for these derivations are based not on each individual patient's oscillometric profile, but on the standard distribution curve or the oscillometric envelope for large, “normal” patient populations. Therefore, such detection thresholds do not take into account biases present in certain patient populations such as patients with heart and circulation problems, arterial sclerosis, arrhythmia, preeclampsia, pulsus alternans, pulsus paradoxus, obesity, and aging populations. These patients can have non-Gaussian distribution curves and their detection threshold values can vary widely compared to the generally accepted thresholds for normal patient populations. As such, the oscillometric NIBP readings for these patients can be incorrect and lead to misdiagnoses of the patient.

### SUMMARY

In one aspect, disclosed is a system including a signal acquisition circuit to acquire a cuff pressure signal using an inflatable cuff. The signal acquisition circuit is also used to generate an oscillometric signal from the cuff pressure signal. The system also includes a user interface to enter one or more patient-specific detection threshold values and a memory to store the cuff pressure signal, the oscillometric signal, and the one or more patient-specific detection threshold values. The system also includes a microprocessor operatively coupled to the signal acquisition circuit, the user interface, and the memory. The microprocessor includes at least one algorithm to use the cuff pressure signal, the oscillometric signal, and the entered one or more patient-specific detection threshold values to calculate patient-specific readings of at least one of mean arterial pressure, systolic blood pressure and diastolic blood pressure.

The one or more patient-specific detection threshold values can be obtained using a non-invasive blood pressure measurement method. The non-invasive blood pressure measurement can be an auscultatory blood pressure measurement method. The one or more patient-specific detection threshold values can also be obtained using an invasive blood pressure measurement. The memory can be to store the one or more patient-specific detection threshold values entered and selectively recall the one or more patient-specific detection threshold values upon command. The system can further include a communication module to receive and transmit wireless signals. The one or more patient-specific detection threshold values can be communicated to a remote database and stored within the patient's individual medical record. The system can be a patient-assigned oscillometric blood pressure monitor system. The system can be an ambulatory oscillometric blood pressure monitor system.

In an interrelated aspect, disclosed is a method including obtaining a reference blood pressure reading of a patient including one or more patient-specific detection threshold values using a first blood pressure measurement method. The method also includes entering the one or more patient-specific detection threshold values into a memory of an oscillometric blood pressure monitor system, storing the one or more patient-specific detection threshold values in the memory of the oscillometric blood pressure monitor system, and calculating at least one of patient-specific mean arterial pressure, systolic blood pressure, and diastolic blood pressure using the one or more patient-specific detection threshold values stored in the oscillometric blood pressure monitor system.

The first blood pressure measurement method can be a non-invasive blood pressure measurement. The non-invasive blood pressure measurement can be an auscultatory blood pressure measurement method. The first blood pressure measurement method can be an invasive blood pressure measurement. Entering the one or more patient-specific detection threshold values into a memory of an oscillometric blood pressure monitor system can include using a user interface on the oscillometric blood pressure monitor system to enter the one or more patient-specific detection threshold values into the memory. Performing a blood pressure reading of the patient using the oscillometric blood pressure monitor system can include acquiring a cuff pressure signal from a signal acquisition circuit using an inflatable cuff to generate an oscillometric signal from the cuff pressure signal. Calculating at least one of patient-specific mean arterial pressure, systolic blood pressure, and diastolic blood pressure using the one or more patient-specific detection threshold values stored in the oscillometric blood pressure monitor system can include using a microprocessor operatively coupled to the signal acquisition circuit and the memory. The microprocessor can include at least one algorithm to use the cuff pressure signal, the oscillometric signal, and the one or more patient-specific detection threshold values to calculate at least one of patient-specific mean arterial pressure, systolic blood pressure, and diastolic blood pressure.

Calculating at least one of patient-specific mean arterial pressure, systolic blood pressure, and diastolic blood pressure using the one or more patient-specific detection threshold values stored in the oscillometric blood pressure monitor system further can include selectively recalling upon command the stored one or more patient-specific detection threshold values. The oscillometric blood pressure monitor system can further include a communication module to receive and transmit wireless signals. The method can further include communicating the one or more patient-specific detection threshold values to a remote database and storing the values within the patient's individual medical record. Entering the one or more patient-specific detection threshold values into a memory can further include receiving a wireless signal from a device used to obtain the reference blood pressure reading. Entering the one or more patient-specific detection threshold values into a memory can further include receiving a wireless signal from the remote database. The oscillometric blood pressure monitor system can be a patient-assigned oscillometric blood pressure monitor system. The oscillometric blood pressure monitor system can be an ambulatory oscillometric blood pressure monitor system.

Articles of manufacture are also described that comprise computer executable instructions permanently stored on non-transitory computer readable media, which, when executed by a computer, causes the computer to perform operations herein. Similarly, computer systems are also described that may include a processor and a memory coupled to the processor. The memory may temporarily or permanently store (e.g., non-transitorily store, etc.) one or more programs that cause the processor to perform one or more of the operations described herein. In addition, methods described herein can be implemented by one or more data processors either within a single computing system or distributed among two or more computing systems.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a schematic representation of an oscillometric blood pressure monitor system according to an implementation;

FIG. 2 illustrates an oscillation envelope indicating theoretical threshold values for systolic and diastolic pressures of a normal patient and a hemodynamically abnormal patient; and

FIG. 3 illustrates a flow diagram illustrating a method to perform a calibration of an oscillometric blood pressure monitor system according to an implementation.

Like reference symbols in the various drawings indicate like elements.

### DETAILED DESCRIPTION

Disclosed herein are NIBP monitor systems, devices, articles, and methods in which a user can adjustably and selectively calibrate to a particular patient threshold values used in the algorithm to assess more accurate systemic and diastolic pressures. The threshold values are programmed based on a reference reading taken by the auscultatory or invasive blood pressure measurement method. The systems and methods described herein compensate for bias present in different patients and patient populations monitored by oscillometric methods of NIBP measurement. The calibrated algorithm can move detection thresholds to the appropriate point on the profile such that subsequent readings will track the blood pressure of the individual patient more accurately. The systems, devices, articles, and methods described herein are appropriate for continuous, automatic NIBP readings and as such can be used for real-time patient monitoring in a variety of medical facilities such as in the hospital ward, operating room, intensive care unit, recovery, and the emergency room. It should be appreciated that the systems, devices, articles, and methods described herein can be used wherever a patient is being treated and should not be limited to a particular medical facility.

Monitor System

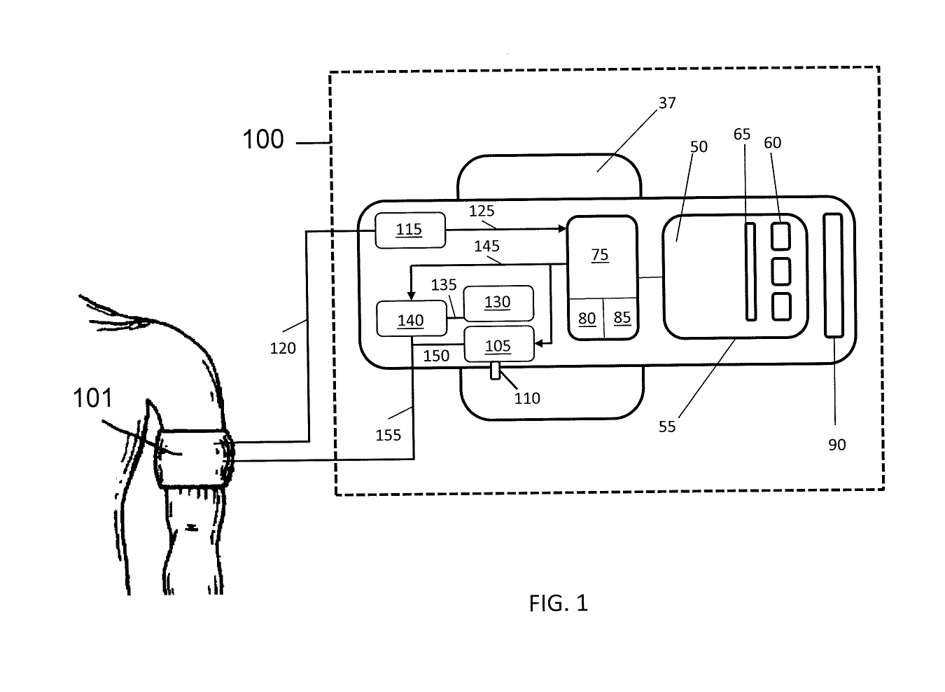

FIG. 1 is a schematic representation of an oscillometric blood pressure monitor system according to an implementation. The monitor system 100 includes a signal acquisition circuit to acquire a pressure signal and generate an oscillometric signal from the pressure signal. The monitor system 100 can be coupled to a cuff 101 to be positioned appropriately on a patient's arm. The cuff 101 can be a conventional flexible inflatable and deflatable cuff 101 that when fully inflated can occlude the brachial artery. The cuff 101 can be deflated using a deflate valve 105 having an exhaust 110 to relieve gradually the arterial occlusion. The deflation of the cuff 101 can occur via a deflate valve 105 controlled by a microprocessor 75.

A pressure transducer 115 can be coupled by a duct 120 to the cuff 101 and used to sense pressure within the cuff 101. As described above, pressure oscillations in the brachial artery can be sensed by changes in the counter-pressure of the cuff 101. These pressure oscillations can be converted into an electrical signal (oscillometric signal) by the pressure transducer 115 and coupled over path 125 to microprocessor 75 for processing. The microprocessor 75 can process the signals from the pressure transducer 115 to produce blood pressure data.

Additionally, a source of pressurized air 130 can be connected via a duct 135 through an inflate valve 140 and a duct 155 coupled to the pressure cuff 101. The inflate valve 140 can be electrically controlled through a connection 145 from the microprocessor 75. The deflate valve 105 can be connected by duct 150 with the duct 155 leading to the cuff 101.

During operation of the system 100 when it is desired to initiate a determination of blood pressure, the microprocessor 75 can provide a signal to open the inflate valve 140 and the deflate valve 105 is closed. Air from the source 130 can be communicated through the inflate valve 140 and duct 155 to inflate the cuff 101 to a desired level. The level is generally above the estimated systolic pressure of the patient. When the pressure in the cuff 101 reaches the predetermined value above the estimated systolic pressure of the patient, the pressure transducer 115 sends a signal on path 125 to the microprocessor 75 indicative of the instantaneous pressure in the cuff 101 to interrupt the inflation of the cuff 101. The signal instructs the inflate valve 140 to close. The deflate routine is commenced such that the blood pressure measurement can be obtained. Upon completion of each measurement cycle, the deflate valve 105 can be re-opened long enough to relax the cuff pressure substantially completely via the exhaust 110. The deflate valve 105 can remain closed ready for the start of a new measurement cycle.

Still with respect to FIG. 1, the microprocessor 75 can include at least one memory 80 coupled to the microprocessor 75 and including at least one program stored thereon. The memory 80 can be any type of memory 80 capable of storing data and communicating that data to one or more other components of the system 100, such as the processor 75. The memory 80 is in communication with the signal acquisition circuit and can store the cuff pressure signals, the oscillometric signals, and one or more threshold values as will be described in more detail below.

The monitor system 100 can include at least one display 50 including a graphical user interface (GUI) 55. The display 50 can provide information to the user such as patient-specific information as well as data being acquired from the patient by the system 100. The display 50 can vary including LCD, LED, plasma, OLED, and the like. The display 50 can be an interactive or touch-sensitive screen having an input device such as a touch screen, a capacitance screen, a resistive screen or the like. The user interface system 55 can include one or more inputs 60 such as fixed buttons associated with fixed functions or changeable functions such as soft keys associated with the display 50. The soft keys can provide functions wherein the function is displayed and the display 50 can change providing different functions in different situations. The fixed input keys can also have a function that changes depending upon the display provided. The inputs 60 can be used, for example, to manually enter reference threshold values. The measurements can also be automatically provided from either another a parameter measurement obtained on the same device, such as an invasive intra-arterial value, or via another device which is wired or wirelessly connected, such as a manual sphygmomanometer. The user interface system 55 can also include one or more indicators and/or alarms 65 that may be visual, auditory through a speaker, tactile, and the like.

The system 100 can include a power system 85. The power system 85 can include a connection to an AC wall power through a power cord. The power system 85 can also include internal battery such as a non-rechargeable or a rechargeable battery. Some embodiments may use a rechargeable battery such as a NiCad battery, LiPo battery, NiMH battery or the like.

The monitor system 100 can be a stationary, portable or telemetry-enabled device. For example, the monitor system 100 can be incorporated into a patient monitor including the Infinity® series of patient monitors including Delta, Delta II, Delta XL, or M540 portable patient monitor (Drager Medical GmbH). The monitor system 100 can include a device that is assigned to a particular patient and located at that patient's bedside. The patient-assigned monitor system 100 can be programmed once for the specific patient to whom the monitor system 100 is assigned. The system 100 can dock with a hardwired docking station 37 located at a patient's bedside and collect data from one or more data acquisition devices besides the NIBP that are additionally acquiring clinical data from a patient. In other implementations, the threshold values can also be programmed into monitor system 100 that is not permanently assigned to a single patient, such as an ambulatory monitor system used to obtain NIBP readings of multiple patients sequentially as will be described in more detail below.

As mentioned above, the monitor system 100 can include a communication module 90 and can communicate with other devices. The communication can be wired or wireless communication capability for the remote sending and receiving of data, such as via WLAN. The communication module 90 can include a transmitter and/or receiver, IEEE 802.11 (WiFi) connection, ZigBee, RFID, infrared, Bluetooth communication device or the like. The system 100 can be in communication via a network of a hospital or other healthcare-providing entity with a hospital information system (HIS). One or more components of the monitoring system 100 can also be in communication with a central patient monitor.

Detection Thresholds

Mean arterial pressure (MAP), systolic blood pressure, and diastolic blood pressure are calculated from the cuff pressure signal, the oscillometric signal and the threshold values. The amplitude of small oscillation signals in the cuff pressure is measured at various cuff pressures where the oscillations are a small fraction of the total cuff pressure. As decrementing continues, the peak amplitudes will normally increase from a lower amount to a relative maximum, and thereafter will decrease. The lowest cuff pressure at which the oscillations have peak amplitude is representative of MAP. As mentioned above, oscillometric NIBP monitors typically record systolic and diastolic pressures at known fractions or detection threshold values relative to the peak amplitude. But as mentioned above, the threshold values can vary patient-by-patient and depending on the patient's health status or patient population. As such, threshold values for one patient or patient population may not accurately determine systolic and diastolic pressures for another patient or patient population.

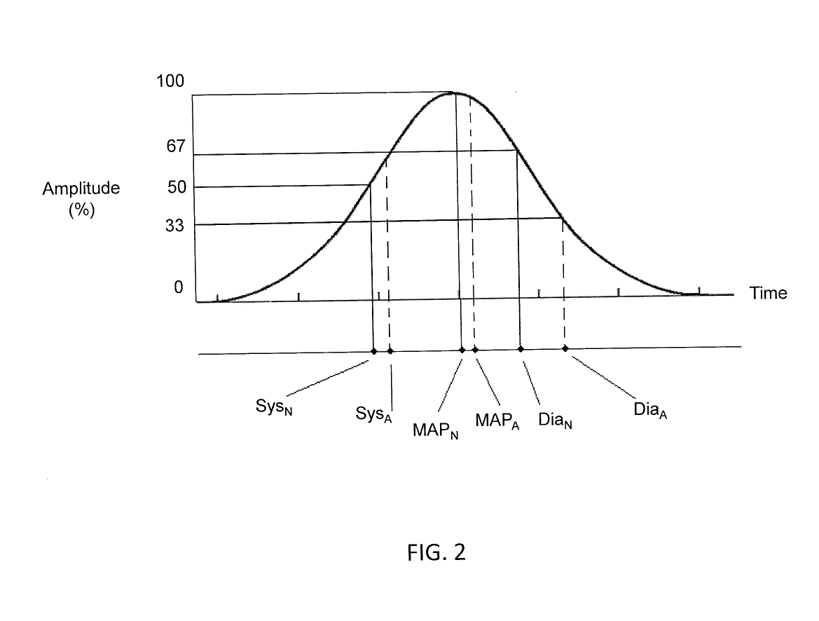

FIG. 2 illustrates an oscillation envelope comparing threshold values for a “normal” patient to that of a patient that is hemodynamically abnormal, for example due to an underlying disease state such as peripheral vascular disease, heart valve stenosis or insufficiency, extreme hypo- or hypertension, or hypertrophy, to name a few. In this example, the MAP for the normal patient (MAP_{N}) is the peak amplitude or 100%, systolic pressure (Sys_{N}) is a 50% threshold value and diastolic pressure (Dia_{N}) is a 67% threshold value. The hemodynamically abnormal patient, in contrast, may have threshold values for systolic pressure (Sys_{A}), mean arterial pressure (MAP_{A}) and diastolic pressure (Dia_{A}) that are shifted towards the right on the curve such that Sys_{A} threshold approaches 67% and Dia_{A} threshold approaches 33%.

A clinician desires assurance that the pressure values obtained by NIBP monitor are within a small window of error. The systems, devices, articles, and methods described herein allow for selecting on a patient-specific basis where on the distribution curve the threshold values land without relying on conventional values.

In use, a user can perform a reference blood pressure measurement of a patient to determine the patient-specific threshold values and where the patient's systolic and diastolic pressures land on the distribution curve. For example, the patient's systolic and diastolic pressures can be shifted to the right on the curve such as shown in FIG. 2. The shifted, patient-specific thresholds obtained from the reference measurement can be used to calibrate a NIBP monitor system 100 assigned to that patient to be used for further blood pressure readings. The calibration of the monitor system 100 can be performed, for example, upon admittance of the patient into a hospital ward. The patient-specific threshold values for systolic and diastolic pressures can be obtained by any method with an inherently higher degree of accuracy, such as the auscultatory measurement method or in some circumstances using an invasive measurement technique, where the accuracy can be validated by the user during the measurement.

The patient-specific threshold values can be programmed into the patient-assigned monitor system 100 such as using inputs 60 of the user interface 55. Alternatively, the patient-specific threshold values obtained can be communicated from the reference blood pressure device to the patient-assigned monitor system 100 without any user input, such as by a wireless method. The patient-specific threshold values can be stored within the memory 80 of the monitor system 100 and incorporated into or used by the program algorithm to determine MAP, systolic and diastolic pressures for each future measurement performed by the NIBP monitor system 100 for that patient. As such, the patient-assigned monitor system 100 is calibrated to the particular patient to whom the monitor system 100 is assigned. NIBP measurements will have a higher accuracy and greater likelihood to detect or diagnose a hypertensive situation for that individual patient for each future reading that is performed using the NIBP monitor system 100. It should be appreciated that the patient-assigned monitor system 100 can be calibrated periodically or according to a pre-set schedule of readings.

In an interrelated aspect, the monitor system 100 need not be assigned to a specific patient to be calibrated to that patient's threshold values. For example, the monitor system can be an ambulatory NIBP monitor system 100 to be used with a plurality of patients. A user can take a reference blood pressure measurement, such as by taking an auscultatory measurement, of a first patient to determine the first patient's specific threshold values. The first patient's individual threshold values for systolic and diastolic pressures can be programmed and stored within the memory 80 of the ambulatory monitor system 100 to calibrate the monitor to that first patient for each future measurement performed by the ambulatory monitor system 100 on that first patient. The same process can be followed in order to calibrate the ambulatory monitor system 100 to each successive patient's threshold values. Each patient's threshold values can be stored within the memory 80 and tagged with patient-specific identification information such that the patient's threshold values can be selectively recalled by the system 100 and used by the system algorithm when another NIBP reading is to be performed for that patient. Alternatively, each patient's threshold values can be communicated to a remote database and stored within a patient's individual medical record. The patient's threshold values can be later selectively recalled and communicated to the monitor system 100 upon command such as prior to a reading using the ambulatory monitor system 100. The recalled patient-specific threshold values can be incorporated into the algorithm prior to obtaining the measurement with the ambulatory monitor system 100. As such that threshold values can be selectable and the algorithm used by the monitor system 100 adjusted on a patient-by-patient basis, for example, as a technician is making the rounds of a series of patients to obtain NIBP measurements. In another implementation, the threshold values used by the algorithm can include user-selectable ranges of threshold values such as for known patient population (e.g. obese, atherosclerotic, preeclampsia, etc.)

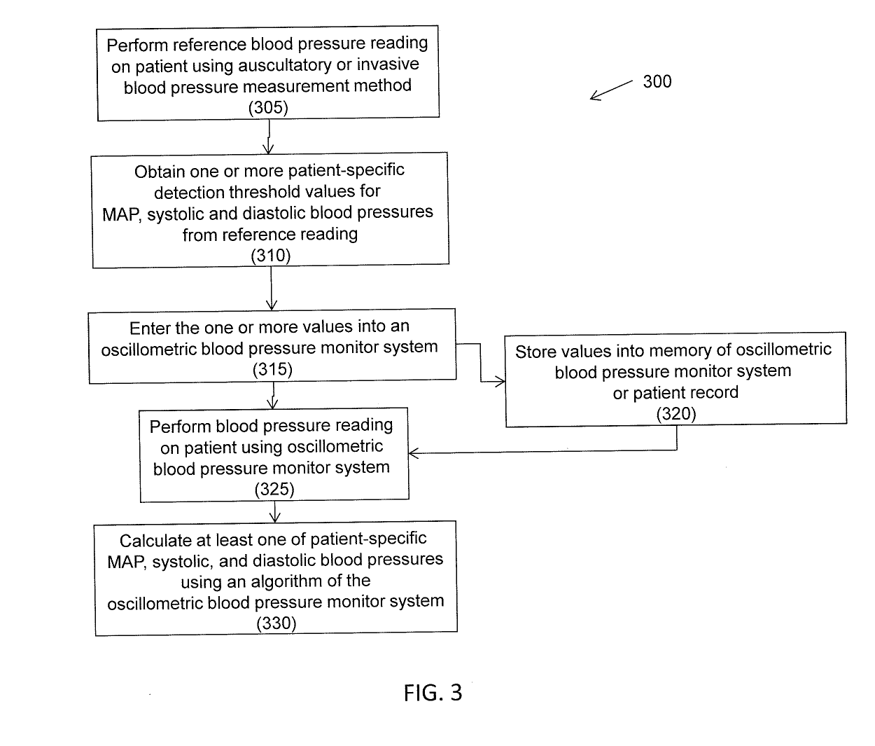

FIG. 3 is a flow diagram 300 illustrating a method to perform a calibration of an oscillometric blood pressure monitor system. A medical professional can perform a reference blood pressure reading on a patient, for example, using an auscultatory blood pressure measurement method or an invasive blood pressure measurement method (305). One or more patient-specific detection threshold values can be obtained for MAP, systolic and diastolic blood pressures from the reference blood pressure reading (310). The one or more patient-specific detection threshold values can be entered into an oscillometric blood pressure monitor system (315). The entered values can be stored into the memory of the oscillometric blood pressure monitor system for future blood pressure readings to be performed by the oscillometric blood pressure monitor system (320). An oscillometric blood pressure reading can be performed on the patient using the oscillometric blood pressure monitor system (325). The reading can be performed automatically by the monitor system or on demand such as by a medical professional or a lay person. The one or more patient-specific detection threshold values stored in the oscillometric blood pressure monitor system memory can be recalled and used to calculate at least one of patient-specific mean arterial pressure, systolic blood pressure, and diastolic blood pressure using an algorithm of the oscillometric blood pressure monitor system (330). It should be appreciated that the entered values need not be stored into the memory of the oscillometric blood pressure monitor system for performing a reading using the device. For example, the entered values can be used immediately by the algorithm of the oscillometric blood pressure monitor system without being stored first. Alternately, the entered values can be stored in a patient database as described above and recalled by the monitor system upon performing a blood pressure reading such that the entered values can be used by the algorithm to calculate the blood pressure values upon taking a blood pressure reading using the oscillometric blood pressure monitor system.

Various aspects of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, the memory, at least one input device, and at least one output device such as a display.

These computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The implementations set forth in the foregoing description do not represent all implementations consistent with the subject matter described herein. Instead, they are merely some examples consistent with aspects related to the described subject matter. Wherever possible, the same reference numbers will be used throughout the drawings to refer to the same or like parts.

Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows and steps for use described herein do not require the particular order shown, or sequential order, to achieve desirable results. Other embodiments can be within the scope of the claims.

1.-23. (canceled)

24. A system, comprising:
a signal acquisition circuit to acquire a cuff pressure signal using an inflatable cuff, and to generate an oscillometric signal from the cuff pressure signal;
a memory to store the cuff pressure signal, the oscillometric signal, and one or more patient-specific detection threshold values; and
a microprocessor operatively coupled to the signal acquisition circuit and the memory, wherein the microprocessor comprises at least one algorithm to use the cuff pressure signal, the oscillometric signal, and the one or more patient-specific detection threshold values to calculate patient-specific readings of at least one of mean arterial pressure, systolic blood pressure and diastolic blood pressure.

25. A system as in claim 24 further comprising:
a user interface coupled to the microprocessor to enter at least one of the one or more patient-specific detection threshold values.

26. A system as in claim 24, wherein the one or more patient-specific detection threshold values are obtained from one or more pressure measurements taken from a specific patient.

27. A system as in claim 24, wherein the one or more patient-specific detection threshold values are obtained using a non-invasive blood pressure measurement method.

28. A system as in claim 27, wherein the non-invasive blood pressure measurement is an auscultatory blood pressure measurement method.

29. A system as in claim 24, wherein the one or more patient-specific detection threshold values are obtained using an invasive blood pressure measurement.

30. A system as in claim 24, wherein the one or more patient-specific detection threshold values are based on a peak amplitude within the oscillometric signal.

31. A system as in claim 24, wherein the memory stores the one or more patient-specific detection threshold values entered and selectively recalls the one or more patient-specific detection threshold values upon command.

32. A system as in claim 24, further comprising a communication module to receive and transmit wireless signals.

33. A system as in claim 32, wherein the one or more patient-specific detection threshold values are communicated to a remote database and stored within the patient's individual medical record.

34. A system as in claim 24, wherein the system is a patient-assigned oscillometric blood pressure monitor system.

35. A system as in claim 24, wherein the system is an ambulatory oscillometric blood pressure monitor system.

36. A method comprising:
obtaining a reference blood pressure reading of a patient comprising one or more patient-specific detection threshold values using a first blood pressure measurement method; storing one or more patient-specific detection threshold values in memory of an oscillometric blood pressure monitor system;
performing a blood pressure reading of the patient using the oscillometric blood pressure monitor system; and
calculating at least one of patient-specific mean arterial pressure, systolic blood pressure, and diastolic blood pressure using the one or more patient-specific detection threshold values stored in the oscillometric blood pressure monitor system.

37. A method as in claim 36 further comprising:
entering the one or more patient-specific detection threshold values into the memory of the oscillometric blood pressure monitor system.

38. A method as in claim 36, wherein the one or more patient-specific detection threshold values are obtained from one or more pressure measurements taken from a specific patient.

39. A method as in claim 36, wherein the first blood pressure measurement method is a non-invasive blood pressure measurement.

40. A method as in claim 39, wherein the non-invasive blood pressure measurement is an auscultatory blood pressure measurement method.

41. A method as in claim 36, wherein the first blood pressure measurement method is an invasive blood pressure measurement.

42. A method as in claim 36, wherein the one or more patient-specific detection threshold values are based on a peak amplitude within the oscillometric signal.

43. A method as in claim 37, wherein entering the one or more patient-specific detection threshold values into a memory of an oscillometric blood pressure monitor system comprises using a user interface on the oscillometric blood pressure monitor system to enter the one or more patient-specific detection threshold values into the memory.

44. A method as in claim 36, wherein performing a blood pressure reading of the patient using the oscillometric blood pressure monitor system comprises acquiring a cuff pressure signal from a signal acquisition circuit using an inflatable cuff to generate an oscillometric signal from the cuff pressure signal.

45. A method as in claim 36, wherein calculating at least one of patient-specific mean arterial pressure, systolic blood pressure, and diastolic blood pressure using the one or more patient-specific detection threshold values stored in the oscillometric blood pressure monitor system comprises using a microprocessor operatively coupled to the signal acquisition circuit and the memory, the microprocessor comprising at least one algorithm to use the cuff pressure signal, the oscillometric signal, and the one or more patient-specific detection threshold values to calculate at least one of patient-specific mean arterial pressure, systolic blood pressure, and diastolic blood pressure.

46. A method as in claim 13, wherein calculating at least one of patient-specific mean arterial pressure, systolic blood pressure, and diastolic blood pressure using the one or more patient-specific detection threshold values stored in the oscillometric blood pressure monitor system further comprises selectively recalling upon command the stored one or more patient-specific detection threshold values.

47. A method as in claim 13, wherein the oscillometric blood pressure monitor system further comprises a communication module to receive and transmit wireless signals.

48. The method of claim 23, further comprising communicating the one or more patient-specific detection threshold values to a remote database and storing the values within the patient's individual medical record;
wherein: entering the one or more patient-specific detection threshold values into a memory further comprises at least one of:
receiving a wireless signal from a device used to obtain the reference blood pressure reading, or
receiving a wireless signal from the remote database.
